(19) United States
(12) Patent Application Publication
(10) Pub. No.: US 2014/0100516 A1
(43) Pub. Date: Apr. 10, 2014

(54) APPARATUS FOR DISINFECTING A SURFACE

(71) Applicants: Allan Kenneth Frazer Grugeon Hunt, Brough (GB); John George Chewins, Andover (GB)

(72) Inventors: Allan Kenneth Frazer Grugeon Hunt, Brough (GB); John George Chewins, Andover (GB)

(73) Assignee: BIOQUELL UK LIMITED, Andover, Hampshire (GB)

(21) Appl. No.: 14/112,687

(22) PCT Filed: May 10, 2012
(86) PCT No.: PCT/GB2012/051022

§ 371 Date: Oct. 18, 2013

(30) Foreign Application Priority Data
May 13, 2011 (GB) 1108141.1

(51) Int. Cl. A61H 33/14

(52) U.S. Cl. CPC A61H 33/14
USPC 604/25

(57) ABSTRACT

The invention relates to improvements in surface disinfection apparatus and in particular to apparatus which may be used to disinfect wounds using high concentration aqueous ozone. The Apparatus comprises application means for applying the fluid to the surface to be treated, fluid delivery means for delivering the fluid to said application means and catchment means for collecting the fluid after it has been applied to the surface. Fluid return means are provided for removing the fluid from the catchment means and means for creating a vacuum in the fluid return means. The apparatus further comprises a releasable coupling having two engagable parts and comprising a plurality of channels, at least one of which channels is connected to the fluid delivery means when the parts are engaged and at least another of which channels is connected to the fluid return means when the parts are engaged. Each channel comprises valve means which open when the engagable parts are engaged to allow fluid to flow through the coupling and are closed when the engagable parts are disengaged to prevent fluid flow. Pressure monitoring means measure the pressure in the fluid return means, wherein the measured pressure is compared to a plurality of pre-set pressure values to determine the correct functioning of the apparatus. If the measured pressure is lower than a first pre-set pressure value fluid is prevented from being delivered to the application means.

The invention relates to improvements in surface disinfection apparatus and in particular to apparatus which may be used to disinfect wounds using high concentration aqueous ozone.

One such system is described in patent specification WO-A-2004/103452 which details an apparatus and method for carrying out the treatment comprising three main components: an apparatus for generating a concentrated aqueous solution of ozone; an apparatus for spraying the ozone solution onto the surface of a limb to be treated; and an apparatus for supporting a limb to be treated and for collecting solution which flows off the treated limb for disposal. The first apparatus comprises a fluid containing tank, coupled to a fluid recirculation loop containing a pumping means and a differential pressure injector. An oxygen source is linked to the differential pressure injector via an ozone generator. Ozone gas is produced within the generator and entrained into the circulating fluid via the differential pressure injector to produce the high concentration aqueous ozone fluid.

The apparatus described in WO-A-2004/103452 preferably provides an aqueous ozone decontamination fluid to a wound of a patient via a multi-phase quick connect coupling described in WO-A-2010/100439. WO-A-2010/100439 details an improvement to the apparatus for supporting a patient's limb and for delivering and collecting solution applied to a patient's limb, wherein a multi-channel coupling is used to allow quick connection and disconnection of the apparatus.

The multi-channel coupling combines a fluid delivery line, an air return line and a fluid return line into a unitary coupling that allows for positioning of the spray means over a patient's wound. A patient's wounded area is placed onto the supporting surface of the treatment tray for application of the aqueous ozone fluid. The patient support function of the tray means that it is preferable from a risk perspective to minimise all forms of electrical contact and conductive paths between the production apparatus and the tray. Eliminating electrical devices or electrical contact from the tray means that it becomes difficult to monitor the functions of the tray. It is important that the tray and its functions are monitored to ensure that a patient is being treated in line with the specified treatment regime. If a tray is not connected or a tray is disconnected during treatment, spraying solution could result in damage to the production system, health and safety risks due to fluid spillage and the uncontained release of ozone gas. The system must also confirm that fluid is being returned to the waste tank of the production unit from the tray. Should the line become blocked, potentially contaminated waste fluid from the patients limb could build-up within the tray, spill over and present health and safety risks.

A pressure sensing means is incorporated into the fluid return line of the aqueous ozone production apparatus, which removes fluid from the patient treatment tray via the quick connect coupling using a vacuum created by a pumping means. The pressure sensing means is used to determine whether the quick connect coupling is connected and hence whether aqueous ozone can be sprayed into the treatment tray. Further, the pressure measuring means can be used to determine whether fluid is being delivered via the fluid delivery channel of the multi-phase coupling into the treatment tray. Still further, the pressure measuring means can be used to determine whether the waste fluid return line of the multi-phase coupling has become blocked.

There is a large amount of prior art related to the application and use of negative pressure therapy devices to treat wounds. Much of this art incorporates the use of a vacuum line and pressure measuring means to control the removal of a fluid from a patient's wound and to detect blockages in the fluid return lines. The prior art does not disclose means for confirming the delivery of a fluid to a patients wound based on monitoring the return line.

JP-A-3051061 describes an apparatus for applying a cleansing solution to a wound via a spray nozzle. Waste liquid is collected in containers via a vacuum line. The patent does not disclose the use of a tray, nor does it collect fluid through a multi-channel coupling.

US-A-2010/0100075 discloses the use of a pressure measuring means in a fluid return line from a patient's wound. The pressure sensor is used to control the vacuum created in the return line to a pre-set level and to determine whether air is leaking into the vacuum area around the patient's wound. The patent does not disclose the use of a fluid delivery line, nor the use of the pressure measuring means to determine its condition.

US-A-2010/0211030 discloses the use of a pressure measuring means in combination with a flow measuring means in a fluid return line from a patient's wound. The software control system of the device uses both sensors to determine the condition of the line (blocked, flowing, etc).

WO-A-2009/019496 discloses the use of a pressure measuring means in combination with a flow measuring means in a fluid return line from a patient's wound. The software control system of the device uses both sensors to determine the condition of the line (blocked, flowing, etc).

The present invention relates to improvements in the usability of this type of apparatus and, in particular, the apparatus described in WO-A-2004/103452.

According to the invention there is provided apparatus for providing a fluid for treating a surface, said apparatus comprising:

application means for applying the fluid to the surface to be treated;

fluid delivery means for delivering the fluid to said application means;

catchment means for collecting the fluid after it has been applied to the surface;

fluid return means for removing the fluid from the catchment means;

means for creating a vacuum in the fluid return means;

a releasable coupling having two engagable parts and comprising a plurality of channels, at least one of which channels is connected to the fluid delivery means when the parts are engaged and at least another of which channels is connected to the fluid return means when the parts are engaged, each channel comprising valve means which open when the engagable parts are engaged to allow fluid to flow through the coupling and are closed when the engagable parts are disengaged to prevent fluid flow;

pressure monitoring means for measuring the pressure in the fluid return means, wherein the measured pressure is compared to a plurality of pre-set pressure values to determine the correct functioning of the apparatus.

If the measured pressure is greater than a first pre-set pressure value fluid is prevented from being delivered to the application means.

If the measured pressure is greater than a second pre-set pressure value but lower than the first pre-set pressure value fluid is delivered to the application means.

Preferably the apparatus further comprises means for providing information to an operator relating to the functioning of the apparatus.

The information provided may indicate whether any of the following conditions apply;

the fluid return means is blocked;

the vacuum creation means is operational;

the engagable parts are engaged; or

the catchment means are connected to the fluid return means.

The fluid is preferably aqueous ozone.

The vacuum creation means is preferably a pump.

The present invention thus uses pressure sensing means in combination with the apparatus identified above, to detect whether the apparatus' are coupled together and whether a fluid is being delivered from one apparatus to another and subsequently returned from second said apparatus to the first.

The invention will now be described, by way of example only, with reference to and as shown in the accompanying drawings, in which:

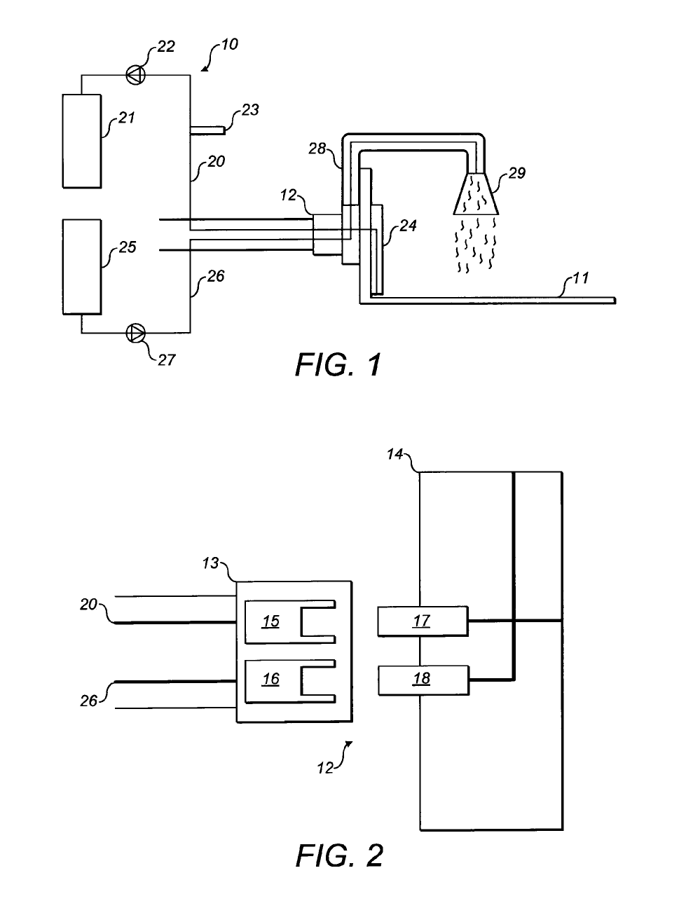

FIG. 1 is a schematic representation of the apparatus of the invention;

FIG. 2 is a schematic representation of the quick connect coupling valved connectors of the apparatus of FIG. 1; and

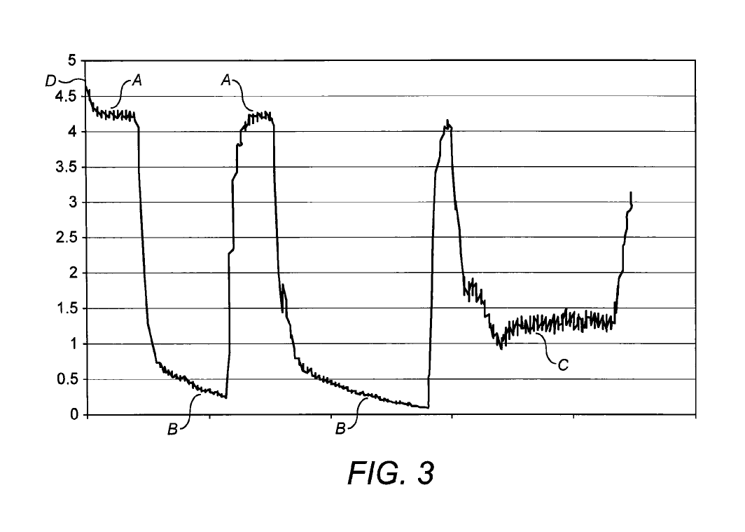

FIG. 3 is a graphical representation of the different pressure levels produced within the waste fluid return line of the apparatus of FIG. 1.

FIG. 1 is a schematic representation of an arrangement that can be used in conjunction with the apparatus described in WO-A-2004/103452 and WO-A-2010/100439.

A patient treatment tray 11 is connected to the aqueous ozone production apparatus 10 (not shown in full) by quick connect coupling 12. The coupling 12 (shown in more detail in FIG. 2) has a clip 13, which is connected to the aqueous ozone production apparatus 10 and a receiver 14, which forms part of the treatment tray 11. The clip 13 contains two female sections 15, 16, and the receiver 14 contains two corresponding male sections 17, 18. Each of the female and male sections 15, 16, 17, 18 are valved to prevent the flow of liquid when disengaged. When the clip 13 is engaged with the receiver 14, female section 15 engages with male section 17 and male section 16 engages with female section 18. The engagement action causes the valves to open, thereby allowing fluid to move through the coupling 12.

Referring back to FIG. 1, a waste fluid return line 20 is connected at one end to coupling 15, within clip 13. At its other end, the waste fluid return line 20 is connected to waste fluid tank 21 via a vacuum producing pump 22 and pressure monitoring means 23. Prior to spraying aqueous ozone solution, the system 10 confirms that patient treatment tray 11 is connected. Pump 22 is activated and a control system (not shown) compares the reading produced by pressure monitoring means 23 against a pre-set pressure level.

If the clip 13 is not connected to the receiver 14, the valves within the male and female sections 15, 16, 17, 18 of the coupling 12 are closed and a large vacuum is created in the waste fluid return line 20. The vacuum is high and the pressure is lower than the pre-set pressure level and hence the system signals the user that the treatment tray 11 is not connected to the apparatus 10, and prevents fluid from being pumped to the treatment tray 11.

If the clip 13 is connected to the receiver 14, the valves within the male and female sections 15, 16, 17, 18 of the coupling 12 are open and pump 22 pulls air from the treatment tray 11 via a fluid pick-up tube 24. The pump 22 is thus not able to generate a large vacuum in line 20 and the pressure remains greater than the pre-set pressure level. The system 10 confirms that the tray 11 is connected and transitions to its next programmed phase.

A high concentration aqueous ozone solution is produced in contact tank 25 and pumped through fluid delivery line 26 by means of a fluid delivery pump 27. The fluid passes through members 16, 18 of the coupling 12 and is routed through spray delivery tube 28 to spray head 29 where it is applied to the surface to be treated. Waste fluid is collected in the base of tray 11 and channelled to the base of fluid pick-up tube 24. Pump 22 creates a vacuum in the waste fluid return line 20 and the waste fluid is taken up through pick-up tube 24, through waste fluid return line 20, to be deposited in waste fluid tank 21.

During the spray phase the pressure within the waste fluid return line 20 is continually monitored by pressure monitoring means 23, such as a pressure transducer. The control system is programmed with a number of pre-set pressure values corresponding to the vacuum levels produced within return line 20 at different operating conditions. These operating conditions are:

High vacuum=blocked waste fluid return line 20 or treatment tray 11 disconnected (no liquid or gas flow within the line 20)

Atmospheric pressure=pump 22 failure (no liquid or gas flow within the line 20)

Low vacuum=no fluid being sprayed into treatment tray 11 or treatment tray 11 connected (gas flow only within the line 20)

Medium vacuum=fluid spraying into treatment tray 11 and being returned to waste tank 21 (normal operation—gas and liquid flow within the line 20)

FIG. 3 shows a graphical representation of the changes in the pressure within waste fluid return line 20, identifying how the different operating conditions can be determined. A timer is started within the control system when the pressure moves outside of the medium vacuum range (normal operation). The pressure within the line 20 must remain at the pre-determined pressure levels for a period of time, such as 30 seconds, before the system 10 will signal to the user. This timer ensures small transient variances in the line pressure do not result in false alarms.

At A the treatment tray 11 is connected and there is only gas flow within the waste fluid return line 20 (low vacuum). At B the treatment tray 11 is disconnected or the waste fluid return line 20 is blocked (high vacuum). At C the treatment tray 11 is connected and liquid is flowing in the waste fluid return line 20 (medium vacuum). At D the pump 22 has failed and no vacuum is created.

1.-7. (canceled)

8. Apparatus for providing a fluid for treating a surface, said apparatus comprising:
application means for applying the fluid to the surface to be treated during an application phase;
fluid delivery means for delivering the fluid to said application means;
catchment means for collecting the fluid after it has been applied to the surface;
fluid return means for removing the fluid from the catchment means;
means for creating a vacuum in the fluid return means;
a releasable coupling having two engagable parts and comprising a plurality of channels, at least one of which channels is connected to the fluid delivery means when the parts are engaged and at least another of which channels is connected to the fluid return means when the parts are engaged, each channel comprising valve means which open when the engagable parts are engaged to allow fluid to flow through the coupling and are closed when the engagable parts are disengaged to prevent fluid flow;
pressure monitoring means for measuring the pressure in the fluid return means, wherein the measured pressure is compared to a plurality of pre-set pressure values to determine the correct functioning of the apparatus;
wherein the apparatus is adapted to prevent fluid from being delivered to the application means if the measured pressure is lower than a first pre-set pressure value prior to the commencement of the application phase.

9. Apparatus as claimed in claim 8, wherein the apparatus is adapted to prevent fluid from being delivered to the application means if the measured pressure after commencement of the application phase falls lower than a second pre-set pressure value, said second pre-set pressure value being lower than the first pre-set pressure value, or rises higher than the first pre-set pressure value.

10. Apparatus as claimed in claim 9 wherein the apparatus is adapted to deliver fluid to the application means if the measured pressure is greater than the second pre-set pressure value but lower than a first pre-set pressure value.

11. Apparatus as claimed in claim 9 further comprising means for providing information to an operator relating to the functioning of the apparatus.

12. Apparatus as claimed in claim 11 in which the information provided indicates whether any of the following conditions apply;
the fluid return means is blocked;
the vacuum creation means is operational;
the engagable parts are engaged; or
the catchment means are connected to the fluid return means.

13. Apparatus as claimed in claim 8 wherein the fluid is aqueous ozone.

14. Apparatus as claimed in claim 8 wherein the vacuum creation means is a pump.
